(19) United States
(12) Patent Application Publication
(10) Pub. No.: US 2007/0071728 A1
(43) Pub. Date: Mar. 29, 2007

(54) Biomimetic nanocomposite

(76) Inventors: Ching-Chang Ko, Eagan, MN (US); Myung C. Chang, Shoreview, MN (US); William H. Douglas, Falcon Heights, MN (US); Wei-Shou Hu, Falcon Hgts, MN (US)

Correspondence Address: FISH & RICHARDSON P.C., PO BOX 1022, MINNEAPOLIS, MN, 55440-1022, US

(21) Appl. No.: 11/305,663

(22) Filed: Dec. 16, 2005

Related U.S. Application Data
(60) Provisional application No. 60/636,611, filed on Dec. 16, 2004.

(51) Int. Cl. A61K 35/12; A61K 38/18; A61K 38/39; A61K 33/42

(52) U.S. Cl. 424/93.7; 424/602; 514/12; 977/906

(57) ABSTRACT

A biomimetic nanocomposite including hydroxyapatite nanocrystals, gelatin, and polymer, wherein the biomimetic nanocomposite is crosslinked is described. Also described is a process for making the nanocomposite. Additionally, a method for using the nanocomposite, and articles formed from the nanocomposite are also described.

### CROSS-REFERENCE TO RELATED APPLICATIONS

This application claims the benefit of U.S. Provisional Application Ser. No. 60/636,611 filed Dec. 16, 2004.

### STATEMENT AS TO FEDERALLY SPONSORED RESEARCH

Funding for the work described herein was provided by the federal government (NIH/NIDCR Grant 1R21 DE015410-01), which may have certain rights in the invention.

### TECHNICAL FIELD

This invention relates to a nanocomposite, and more particularly to a biomimetic nanocomposite.

### BACKGROUND

Many different materials have been used for bone replacement and substitutes. However, the materials used have not performed as well as natural bone. These bone substitutes have not been ideal because they have very different mechanical properties and often exhibit less than desirable biocompatibility.

Attempts at bone replacement have used a variety of foreign materials, with resulting associated problems. Metals that have been used to replace bone structure, such as stainless steel and titanium, have been found to mechanically mismatch with properties of bone to which they are implanted or attached. Additionally, these materials often cause allergic reactions and inflammation due to abrasive particles and leached ions such as Nickel, Cobalt, Chromium, Aluminum, and Vanadium ions. Teflon joint implants have been used, but have been known to shatter and erode when used in applications requiring lots of repetition and force, such as use as jaw implants. Bio-inert materials such as alumina and zirconia ceramics exhibit many of the same clinical problems associated with metal implants.

Other approaches have used many of the same materials as found in natural bones in an attempt to create more viable and long lasting bone replacement materials. Natural bones are an extracellular matrix mainly composed of hydroxyapatite crystals and collagen, with the hydroxyapatite well-mineralized on collagen at body temperature. The strength of the hydroxyapatite/collagen bonding and the quality and maturity of the collagen fibers are important for the mechanical properties of bone. Therefore, many of these attempts have focused on developing hydroxyapatite and collagen mixtures for bone substitutes. However, collagen is an expensive material, and the reaction of collagen with hydroxyapatite can be difficult to control. This lack of control has led to materials having reduced and/or inconsistent physical strength.

Implants using cement and ceramic materials, such as calcium phosphate, have also been made. These cements and ceramics overcome many of the problems noted above, as they can directly connect with bone and do not exhibit the reactions and inflammation common to many other implants. Additionally, as these materials are biocompatible, natural bone material grows slowly into the implants over time. However, these cements and ceramics are brittle, often have poor flexture strength, and are weak in energy absorption. Also, the materials used have generally been difficult to sculpt, leading to problems with irregular defects, and granule migration from the implant site. Therefore, these materials have not been widely used, and when used, have generally been limited to non-load bearing indications.

Natural bone, either large pieces or compositions, have also been used, with compositions using aggregates of bone particles receiving a high level of interest. The objective has been to more closely mimic natural bone and increase the strength of the implant. This also retains biocompatibility and allows bone ingrowth and assimilation. However, there are problems with harvesting and availability of bone components. Additionally, there are risks and complications associated with bone grafts or compositions, including risks of infection, viral transmission, disease, rejection, and other immune system reactions.

In addition to bone replacement, attempts have also been made to replace other bodily tissues. Various attempts have used animal tissues to replace human tissues, have used tissues from other locations in the body, or have attempted to use synthetic materials. These methods all have associated drawbacks and shortcomings.

### SUMMARY

Therefore, there exists a need for a synthetic implant material that is strong, cost-effective, and which offers a high degree of biocompatibility, while exhibiting rapid integration with the surrounding tissues and structures.

In one aspect, a biomimetic nanocomposite including hydroxyapatite nanocrystals, gelatin, and polymer, wherein the biomimetic nanocomposite is crosslinked is described.

In another aspect, a method for producing a biomimetic nanocomposite, including mixing calcium hydroxide, phosphoric acid, and gelatin under aqueous conditions, co-precipitating the mixture, adding a polymer, and adding a cross-linking agent is described.

In another aspect, a method of using a biomimetic nanocomposite is described, including implanting an article comprising a biomimetic nanocomposite, wherein the biomimetic nanocomposite includes hydroxyapatite nanocrystals, gelatin, and polymer, and wherein the biomimetic nanocomposite is crosslinked.

In another aspect, a process for forming a polymerization matrix is described, including using a gelatin as an embedding media for the mineralization of hydroxyapatite nanocrystals, adding a polymer, and adding a crosslinking agent.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a representation of an embodiment of a biomimetic nanocomposite.

FIG. 2 is a flowchart showing process steps for producing a biomimetic nanocomposite.

FIG. 3 is a series of photographs showing TEM morphology and ED pattern for hydroxyapatite coprecipitation slurry after coprecipitation reactions at various temperatures.

FIG. 4 is a photograph of the results of a seeding of CHO cells on a biomimetic nanocomposite disc after 9 days of incubation.

FIG. 5 is a photograph of the results of a seeding of osteoblast cells on a biomimetic nanocomposite disc after 12 days of incubation.

FIG. 6 is a photograph of the results of a seeding of bone marrow stem cells on a biomimetic nanocomposite disc after 3 days of incubation.

FIG. 7 is a scanning electron microscope (SEM) image of the results of a seeding of osteoblast cells on a biomimetic nanocomposite disc after 9 days of incubation.

### DETAILED DESCRIPTION

Biomimetic processing is based on the idea that biologic systems store and process information at the molecular level. The extension of this concept to the processing of synthetic bone and other synthetic tissues has increased in the last few years.

A biomimetic nanocomposite is described that can be used as a replacement material for a variety of body applications. The biomimetic nanocomposite includes a fully intermixed and nearly uniformly dispersed composition including hydroxyapatite nanocrystals, gelatin fibers, and a polymer. The biomimetic nanocomposite is also crosslinked.

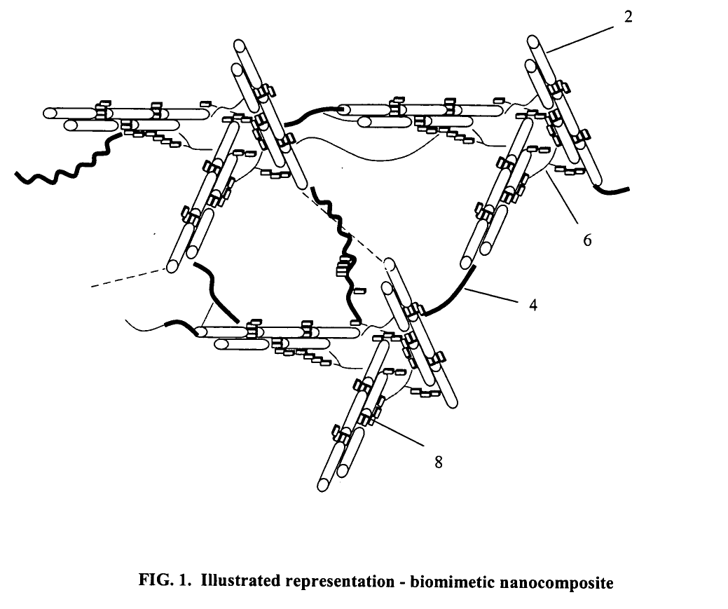

As shown in the representation of FIG. 1, hydroxyapatite nanocrystals 8 are embedded into a matrix formed by polymer chains 4 and gelatin fibers 2. A crosslinking agent has been used to crosslink the materials. After crosslinking, the used crosslinking agent 6 remains in and contributes to the matrix. All of the components are fully intermixed and nearly uniformly dispersed, resulting in relatively consistent properties throughout the composition. The structure is formed by gelatin fibers 2, which are intermixed with the polymer chains 4, and are assisted in being held together and crosslinked via the crosslinking agent 6. The hydroxyapatite crystals 8 are set into, upon, and held by this structure.

One component is gelatin. It has been found that gelatin can provide a bioactive surface to induce hydroxyapatite crystal growth. Suitable gelatins include both high bloom and low bloom gelatin. Preferably, gelatins having a bloom value between about 100 and about 300 will be used. Bloom value is a measurement of the strength of a gel formed by a 6 and ⅔% solution of the gelatin, that has been kept in a constant temperature bath at 10 degrees centigrade for 18 hours. The properties of the final biomimetic nanocomposite depend in part on the characteristics of the gelatin used. An example of a suitable gelatin is standard unflavored gelatin (available from Natural Foods Inc., Canada). The gelatin may be dissolved into solution before use. Preferably, the gelatin will be dissolved to form an aqueous solution. The gelatin may be used without purification or other prepatory steps.

Variously, gelatin may be obtained that is produced from different animals, including cows and pigs. Gelatin may be produced from various body parts, including bone and skin. The gelatin may be selected according to the desired application, as different gelatins may provide a better choice for the composite, depending upon the desired mechanical properties or biological activity level. Generally, it have been found that bovine gelatin provides better composites for many applications.

Additionally, the gelatin may be modified prior to use in a reaction mixture. Preferably, the gelatin will be phosphorylated before use in the reaction to form the biomimetic nanocomposite. For example, the gelatin may be phosphorylated by the addition of phosphoric acid (available from chemical supply firms such as Fisher Scientific and Sigma Chemical) to a gelatin solution, or the gelatin may be added to a phosphoric acid solution. It is believed that phosphorylation leads to and enables better dispersion and growth of the hydroxyapatite nanocrystals. In solutions with phosphorylated gelatin, there will typically be excess phosphoric acid available for later crystal formation and/or growth.

Calcium hydroxide is available from chemical supply firms such as Fisher Scientific and Sigma Chemical. However, calcium hydroxide may also be produced in a process including calcining calcium carbonate, which removes carbon dioxide to form calcium oxide. After calcining, the calcium oxide is hydrated to form calcium hydroxide. Following hydration, the calcium hydroxide may be weighed as a quality check. Due to the reactive nature of calcium hydroxide, and the tendency of calcium hydroxide to degrade quickly, special care should be taken with calcium hydroxide to ensure a high quality level of the calcium hydroxide. Because of this concern with the quality of the calcium hydroxide, producing calcium hydroxide just prior to use is preferred.

The hydroxyapatite nanocrystals are formed through a reaction between phosphoric acid or phosphorylated locations on the gelatin fibers and calcium hydroxide. The phosphorylated locations are frequently the starting locations for hydroxyapatite crystal growth. However, hydroxyapatite crystal growth may also occur in solution between the phosphoric acid and calcium hydroxide components. These crystals may grow and embed themselves into the matrix structure. These crystals may bind themselves to groups, such as carboxyl and amide groups, on the gelatin molecules. Once begun, the crystals grow by incorporating more calcium hydroxide and phosphoric acid components into the crystal.

A polymer is used to help produce a matrix for the biomimetic nanocomposite. Polymers, such as polyacrylic acid, may be purchased from chemical supply firms such as Fisher Scientific and Sigma Chemical. Alternatively, a polymer may be produced from suitable polymerizable components, which are also available from the same chemical supply firms. Suitable polymerizable components include, but are not limited to, acrylic acid, methacrylic acid, amides, vinyls, and combinations thereof. The polymer may be formed from polymerizing the polymerizable components. Examples of suitable polymers include polyacrylic acid (PAA), polymethacrylic acid (PMA), polyamide (PA), polylactic acid (PLA) and polyvinyl alcohol (PVA). Preferably, the polymer used will be a polymerized acid. More preferably, the polymer used will be PAA. The polymer may be a bioabsorbable polymer. The polymer may be a biodegradable polymer. The polymer may be a hydrophilic polymer.

A crosslinking agent is used to help bind and hold the matrix structure together. Suitable crosslinking agents include those that can assist in creating a matrix with the other components. Examples of suitable crosslinking agents include glutaraldehyde (GA), multi-functional aldehydes, Ethylene Glycol Diglycidyl Ether(EDGE), and mixtures thereof. Preferably, glutaraldehyde or a variant thereof will be used. Glutaraldehyde is a dialdehyde, and generally acts to stabilize structures by rapid cross-linking.

The mineralized gelatin fibers are crosslinked with the polymer, forming a nearly uniformly dispersed biomimetic nanocomposite. The microstructure contains hydroxyapatite nanocrystals along the gelatin fibers. The properties of the resulting biomimetic nanocomposite can vary widely, based in part on the different amounts of polymer and crosslinking agent used in forming the biomimetic nanocomposite.

Optionally, other components or additives may be added to the biomimetic nanocomposite. These additives may be added for various reasons. For example, additives may be added to increase biocompatibility or to decrease the possibility of rejection. Additives may be added to decrease the risk of infection, to increase the rate of natural bone growth in the biocompatible nanocomposite, or to increase the rate of natural cell growth near the implant. Additives may be added to change or enhance some of the properties of the biomimetic nanocomposite. Additionally, the biomimetic nanocomposite may include additives for other purposes. Examples of suitable additives include growth factors, cells, other materials and elements, curing or hardening components, and other possible additives.

Optionally, growth factors may be added to the biomimetic nanocomposite. Among other benefits, growth factors can assist in increasing natural growth, including the growth of natural tissues and bone into the area of the biomimetic nanocomposite. Examples of suitable growth factors include bone morphogenic protein (BMP), transforming growth factor (TGF-β), vascular endothelial growth factor (VEGF), matrix gla protein (MGP), bone siloprotein (BSP), osteopontin (OPN), osteocacin (OCN), insulin-like growth factor (IGF-I), or procollagen type I (Pro COL-α1).

Optionally, cells may be added to the biomimetic nanocomposite. Cells may be added to the biomimetic nanocomposite in order to increase the rate of natural bone growth in the area of the biomimetic nanocomposite. Also, precursor cells may be added to the biomimetic nanocomposite to speed the rate of natural cell growth. Suitable cells include, but are not limited to, osteoblasts, osteoclasts, osteocytes, and stem cells.

Optionally, other materials and elements may be added to the biomimetic nanocomposite. Elements and materials may be added to provide an additional feature, property, or appearance to the biomimetic nanocomposite, or for other reasons. Examples of suitable elements include fluoride, calcium, ions thereof, or other elements or ions. Examples of other suitable materials include polymers, ceramic particles, radio-opaque components, metals, and other materials. Variously, the biomimetic nanocomposite can include ceramic particles, fluoride, calcium, and/or a radio-opaque material.

Optionally, curing additives may be added to the biomimetic nanocomposite. Suitable curing agents include photo- and uv-curable agents. A curing agent enables the biomimetic nanocomposite to harden more rapidly and allows the biomimetic nanocomposite to be used for a wider variety of uses. For example, a paste or viscous mixture of the biomimetic nanocomposite could be applied to an area of a bone or a tooth, and then rapidly cured to harden in place. This approach has the potential to improve the outcome and decrease patient recovery time.

Examples of other optional additives include growth inhibitors, pharmaceutical drugs, anti-inflammatory agents, antibiotics, and other chemicals, compositions, or drugs. These could be used in various applications of the biomimetic nanocomposite. For example, growth inhibitor may be used the prevent the ingrowth of certain undesirable cells, so that the biomimetic nanocomposite continues to function most effectively. Antibiotics may be used to decrease the likelihood of infection around the area of treatment. Pharmaceutical drugs, anti-inflammatories, and antibiotics may be used to reduce inflammation, minimize bleeding, increase healing, or for other uses.

The biomimetic nanocomposite may be used for a wide range of alloplastic uses, for a variety of purposes, and in a variety of applications. Alloplastic refers to synthetic biomaterials, in contrast to natural biomaterials which may be from the same individual (autogenic), from the same species (allogenic), or from a different species (xenogenic). The properties of the biomimetic nanocomposite may be modified to better meet the requirements of the use, purpose, or application for which it is intended. The properties depend in part on the gelatin used, the alignment of fibers and chains, the amount and type of polymer used, and the amount and type of crosslinking agent used. Thus, the resulting biomimetic nanocomposite may have a wide range of mechanical properties. The malleability may range from very stiff to very rubbery. For example, in general if a large amount of polymer is used, with no overall alignment of the fibers and chains predominating, the resulting biomimetic nanocomposite is very stiff and can be used as alloplastic grafts in load bearing areas, such as for bone replacement. As another example, generally using lesser amounts of polymer with a strong alignment of fibers and chains results in a soft, rubbery biomimetic nanocomposite that can be used as alloplastic grafts in areas with frequent flexion, including uses such as cartilage or ligament replacement. Other combinations, using various amounts of polymer, crosslinking agents, and gelatin, and having various amounts of alignment of fibers and chains, lead to biomimetic nanocomposites having different properties. These various properties lead to the ability of the biomimetic nanocomposite to be used for many additional types of alloplastic grafts and/or replacement materials, including uses such dental implants, joints, etc. In addition, the biomimetic nanocomposite has other additional valuable properties. For example, the biomimetic nanocomposite is resistant to crack propagation in both dry and wet states.

The biomimetic nanocomposite can also be used in a wide range of tissue engineering applications. The biomimetic nanocomposite can be made in scaffolds, which can deliver cells, growth factors, and other additives to a healing site. This can be used to regenerate bone, cartilage, and other tissues. Nano-scaled microstructures can be used to promote cell attachment, growth, and differentiation.

Using a frame structure, or other types of open structures, may be a valuable approach for many applications. Cells, drugs, and other materials may be seeded into or added to the frame to promote the growth of natural cells. This may encourage and promote the integration of the biomimetic nanocomposite with the natural tissues of the body. Through the growth of natural cells and bioabsorption of the biomimetic nanocomposite, a more natural and effective approach may be taken. Thus, tissue engineering may be used to replace or augment many natural body tissues. Tissues may be regenerated using these types of structures, and additives may be used to compensate for deficiencies in the patient. Other structures that promote the rapid integration of the biomimetic nanocomposite with the natural tissues may also be used effectively. For example, a structure of the biomimetic nanocomposite may be implanted into a bone, which then acts to stimulate bone regeneration. As another example, the biomimetic nanocomposite may be implanted for cartilage replacement, which may stimulate cartilage regeneration.

The biomimetic nanocomposite may be produced in different forms, depending upon the intended use and purpose. Suitable forms include solid, putty, paste, and liquid. If the biomimetic nanocomposite is in solid form, it may be, for example, be a shaped or unshaped solid, it may be a pre-formed solid, it may be a frame or a lattice, or another solid form. The biomimetic nanocomposite may be formed into a porous scaffold. The solid form may be very stiff, stiff, slightly flexible, soft, rubbery, or other. The biomimetic nanocomposite may be a putty. If in putty form, it may be anywhere from a dense or thin putty. The biomimetic nanocomposite may be a paste. If a paste, it may be anywhere from a thick to a thin paste. If a liquid, it may be from very viscous to very thin.

Due to the wide range of forms in which the biomimetic nanocomposite may be produced, the biomimetic nanocomposite lends itself to a wide range of uses. The biomimetic nanocomposite may be used with bones, such as for bone graft material or as bone cement. The biomimetic nanocomposite may be used for dental procedures, such as for dental implants, fillings, jaw strengthening or replacement, or joint replacement. The biomimetic nanocomposite may be used for cartilage replacement or reinforcement. The biomimetic nanocomposite may be used for tendon or ligament replacement or repair. The biomimetic nanocomposite may also be used in a wide range of tissue engineering applications, including assisting in regenerating bodily tissues.

There are two major types of bone material—spongy bone and compact bone. Compact bone is also sometimes known as lamellar or cortical bone. The basic units of compact bone are tightly packed plates wound into tubular forms, called osteons. Each osteon has a capillary running through its central channel. The osteons are arranged in vertical stacks to form a hard, shell-like membrane. Spongy bone is also sometimes known as trabecular or cancellous bone. Spongy bone comprises millions of tiny formations that form a lattice-like matrix.

Most bones contain both compact and spongy bone tissue. Generally, compact bone forms the dense outer casing, providing the majority of the bone strength and structure, while spongy bone spans the interior. Depending upon the bone, the proportion of compact bone and spongy bone can vary. Long, regular bones, like those of the arms, legs, and ribs, are composed primarily of compact bone. Irregularly shaped bones, such as the heads of the leg bones, the pelvis, and the vertebrae, are composed principally of trabecular bone.

One application of the biomimetic nanocomposite is to replace bone material in the body. The biomimetic nanocomposite may have properties similar to natural bone. For example, a biomimetic nanocomposite may have similar strength modulus to natural bone. The benefit of having a similar strength modulus is that biomechanical mismatch problems, such as stress shielding, can be minimized. Nanoindentation is a mechanical microprobe method that enables the direct and simultaneous measurement of strength modulus and hardness. The resolution of the test method enables the measurement of bones and materials at a very fine level. Nanoindentation is discussed in more detail in Ko, C C et al., Intrinsic mechanical competence of cortical and trabecular bone measured by nanoindentation and microindentation probes, Advances in Bioengineering ASME, BED-29:415-416 (1995). The test may be conducted using an MTS nanoindenter XP (available from MTS Systems Corporation, Eden Prairie, Minn.). The method used may be as described in Chang M C et al., Elasticity of alveolar bone near dental implant-bone interfaces after one month's healing, J. Biomech. 36:1209-1214 (2003).

Additionally, the compressive strength of the biomimetic nanocomposite and various natural bones may be tested and compared. A biomimetic nanocomposite may have compressive strength comparable to that of natural bone. A compressive strength test may be conducted using an Instron 4204 Tester (available from Instron Corporation, Canton, Mass.). Tests are conducted according to ASTM C39 “Standard Test Method for Compressive Strength of Cylindrical Concrete Specimens,” and may include using cylindrical samples with height to diameter ratio of 2:1.

The biomimetic nanocomposite may be used as alloplastic bone graft material. The biomimetic nanocomposite may form an article for use in bone replacement. In addition to bone replacement, the biomimetic nanocomposite may be used to replace, repair, support or reinforce other body tissues, including tendon, cartilage, ligament, tooth, and other tissues. For example, the biomimetic nanocomposite may form an article for use in tissue engineering.

An implant may be formed of the biomimetic nanocomposite. A biomimetic nanocomposite implant may be used for bone replacement. A biomimetic nanocomposite implant may be used for tooth replacement. A biomimetic nanocomposite implant may be used for cartilage replacement. In addition, a biomimetic nanocomposite implant may be used for other uses such as described herein.

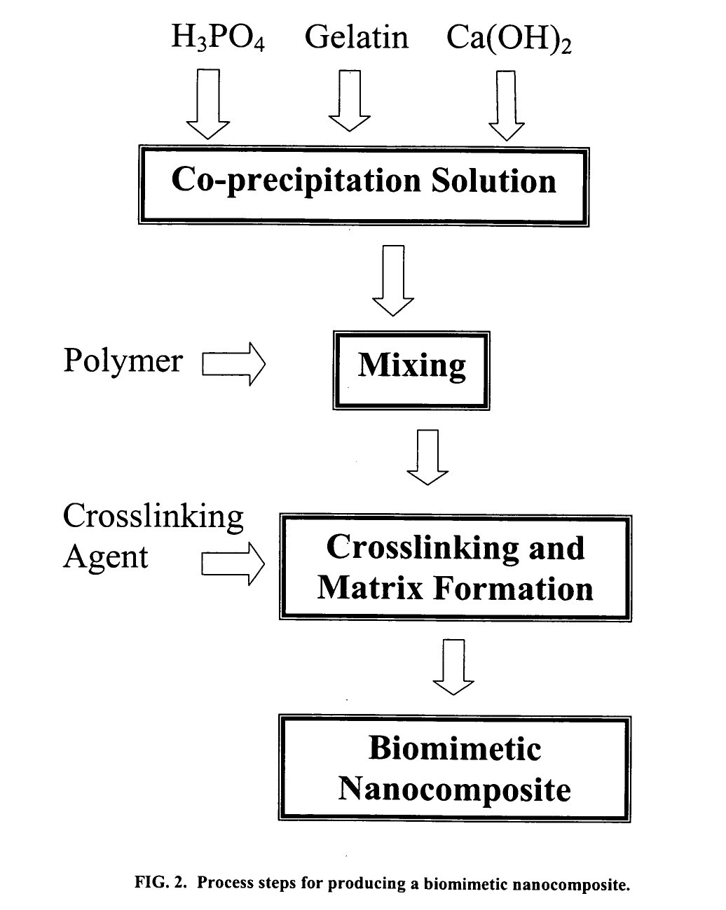

A method for producing a biomimetic nanocomposite is also described. A flowchart diagram including the major process steps for making a biomimetic nanocomposite is shown in FIG. 2. As can be seen, the process does not require isostatic presses to increase the density of the composition, nor a lengthy double diffusion process.

A reactor is setup with temperature control and stirring. A mixture of calcium hydroxide, phosphoric acid, and gelatin (GEL) is mixed together using a high degree of agitation. These components should be as pure as possible to minimize any contaminants which might weaken the resulting nanocomposite. Purchased or produced, the components will preferably be placed into solution prior to use. More preferably, the components will be in an aqueous solution. The various components may be added all at once, or may be added gradually. If added gradually the components in solution may be added using pumps, such as peristaltic pumps (such as Masterflex, available from Cole-Parmer).

The gelatin may be added separately, or may be pre-mixed together with one of the other components prior to addition. Preferably, the gelatin will be pre-mixed with the phosphoric acid in order to phosphorylate the gelatin. This has been found to lead to better dispersion and growth of the nanocrystals. The gelatin may be dissolved in a solution, and the phosphoric acid added to the solution, or the gelatin may be added to the phosphoric acid. Preferably, the gelatin will be added to and dissolved in an aqueous solution of phosphoric acid. In order to assist in dissolving the mixture, the temperature may be controlled between about 35° C. and 40° C., and the mixture stirred during the addition and dissolving. A wide range of gelatin concentrations may be used. Preferably, the concentration will be greater than about 0.001 mmol, greater than about 0.01 mmol, or greater than about 0.025 mmol. Preferably, the concentration will be 100 mmol or less, 10 mmol or less, or 1 mmol or less.

In order to enable sufficient phosphorylation of the gelatin, this mixing should continue for some time. Suitably, the mixing will continue for at least about 2 hours. Preferably, the mixture will be mixed for at least about 5 hours. Suitably, the mixing will be continued for less than about 24 hours. Preferably, the mixing will continue for less than about 18 hours, and more preferably less than about 12 hours. It has been found that insufficient mixing time leads to less than a desirable amount of phosphorylation, and results in larger, less well-dispersed crystals later in the process. When mixed for longer periods, the gelatin begins to lose the ability to react with the other components, with the result that the crystals are no longer held as well by the gelatin later in the process. The ability to hold the crystals and coordinate the gelatin with the hydroxyapatite (HAp) continues to decline with time, until it decreases sharply after 24 hours of mixing. The obtained intermediate slurries have been found to show different qualities and gelling status based on the phosphorylation time.

After preparation, the calcium, phosphoric acid, and gelatin components are added together, using agitation and while controlling the pH and temperature. As the components streams are added, a co-precipitation begins to occur. This begins to form hydroxyapatite in the gelatin by the co-precipitation of the calcium and phosphate ions from the components. This co-precipitation results in the formation of hydroxyapatite (HAp) nanocrystals in the gelatin. Preferably, the conditions and component concentrations are maintained such that the continued high-speed agitation and controlled conditions result in the continued formation of hydroxyapatite nanocrystals, rather than the growth of macro-crystals. Under high agitation, this mixture forms a slurry.

During addition of the components as well as during agitation, the pH of the mixture may be controlled. Suitably, the pH will be controlled to be greater than about 7.0, preferably greater than about 7.5, and more preferably greater than about 7.8. Suitably, the pH will be controlled to be less than about 9.0, preferably less than about 8.5, and more preferably less than about 8.2. The pH control may be controlled using the components of the reaction process, using means known in the art. For example, a pH controller (such as Bukert 8280H, available from Bukert) may be used to measure the pH and control the action of the pumps used to add the various components.

The temperature of the mixture may also be controlled during addition of the components and during agitation. Preferably, the temperature will be controlled using a water bath (available from Boekel), though many other means of temperature control are also suitable. Suitably, the temperature will be controlled to be greater than about 30° C., preferably greater than about 34° C., more preferably greater than about 36° C. Suitably, the temperature will be controlled to be less than about 48° C., preferably less than about 45° C., and more preferably less than about 40° C. At too low of a temperature, there is insufficient energy to lead to good crystal growth. At too high of a temperature, the crystals grow larger than the desired size.

The co-precipitation is characterized by being a low cost, simple process which is easily applicable and adaptable to industrial production. Moreover, the hydroxyapatite crystals prepared by the co-precipitation generally have the benefits of very small size, low crystallinity, and high surface activation. This enables the biomimetic nanocomposite to meet many different demands.

Properly controlled, the co-precipitation results in a uniform dispersion of hydroxyapatite nanocrystals. Suitably, calcium and phosphate will be present in sufficient amounts to enable the formation and growth of hydroxyapatite nanocrystals. Preferably, the ratio of the number of moles of calcium to the number of moles of phosphate present will be from about 1.5 to about 2.0, more preferably present in a ratio from about 1.6 to about 1.75, and most preferably from about 1.65 to about 1.70. The nanocrystals formed may be needle-shaped, plate-shaped, or may have other crystal shapes. Preferably, hydroxyapatite crystals formed will be needle-shaped.

After addition of all of the components into the co-precipitation reaction, agitation is stopped. The slurry is collected after 24 hours without agitation. The slurry may be collected using various approaches, but preferably will be collected by vacuum filtration.

The collected slurry is transferred to another reaction flask, setup with high-speed stirring and temperature control. One or more polymers is added to the flask with vigorous stirring. Preferably, the polymers will be added as a solution of one or more polymers. The polymer may be purchased or may be produced from polymerizable components. Suitable polymerizable components include, but are not limited to, acrylic acid, methacrylic acid, amides, vinyls, and combinations thereof. Examples of suitable polymers include polyacrylic acid (PAA), polymethacrylic acid (PMA), polyamide (PA), polylactic acid (PLA) and polyvinyl alcohol (PVA). Preferably, the polymer used will be a polymerized acid. More preferably, the polymer used will be PAA. The polymer may be a bioabsorbable polymer, a biodegradable polymer, and/or a hydrophilic polymer.

The polymer may be added in various amounts, depending upon the desired properties of the biomimetic nanocomposite, and the concentration of the other components. The polymer may be added directly, or more preferably, will be added as an aqueous solution or mixture. The amount will be selected in order to assist in achieving a biomimetic nanocomposite having the desired properties. For example, typically, a polymer solution will be created having a polymer concentration from about 0.00001 mol/liter to about 0.01 mol/liter.

The polymer may be added to the other components all at once or over a period of time. A sufficient amount of polymer will be added to assist in forming the desired matrix. The component mixture continues to be stirred during addition of the polymer, and following addition of the polymer, the mixture is stirred for a sufficient time while maintaining the temperature. The temperature will suitably be greater than about 30° C., greater than about 34° C., and more preferably greater than about 36° C. The temperature will suitably be less than about 48° C., preferably less than about 45° C., and more preferably less than about 40° C.

After addition of the polymer, one or more crosslinking agents is added to the flask with vigorous stirring. Preferably, the one or more crosslinking agents will be placed into solution prior to addition. Depending upon the desired properties of the biomimetic nanocomposite, and the concentration of the other components, the crosslinking agent may be added in various amounts. The amount will be selected in order to assist in achieving a biomimetic nanocomposite having the desired properties. For example, typically, a crosslinking agent solution will be created having from about 0.01% to about 1.0% by weight crosslinking agent. Preferably, the solution will have a concentration from about 0.05% to about 0.1% by weight crosslinking agent, and more preferably the solution will have a concentration from about 0.07% to about 0.09% by weight crosslinking agent. Suitably, sufficient crosslinking solution will be added to crosslink the other components and help form the composition matrix. The crosslinking agent may be added all at once, or may be added gradually. Preferably, the crosslinking agent will be added gradually, using a pump. Examples of suitable cross-linking agents include GA, multi-functional aldehydes, EDGE, and variants and mixtures thereof. Preferably, glutaraldehyde or a variant thereof will be used.

Alternatively, the polymer and crosslinking agent may be added simultaneously or near-simultaneously, and such addition may be gradual or rapid. During addition, the mixture should be vigorously stirred or agitated.

After addition of the crosslinking agent, the high-speed agitation is continued. This maintains the distribution of the components evenly throughout the mixture. The crosslinking agent assists in linking the components together, holding the various components together, and forming a three dimensional matrix. The matrix is formed by a structure including the HAp/GEL composite and the polymer, crosslinked via a cross-linking agent. The agitation continues for long enough to ensure complete mixing. Suitably, the time will be more than about 10 minutes, and preferably more than about 15 minutes of mixing after addition is complete.

Then, the resulting product is collected. It may be collected using various means, and preferably vacuum filtration will be used. The collected composite may then be stored for later use, or may be dried. Alternatively, a structure may be formed using vacuum filtration to make a sample body which may then be dried. Abundant ion-exchanged, double-distilled water may be used to wash the biomimetic nanocomposite prior to drying.

A product or shape may be formed from the damp biomimetic nanocomposite, or the biomimetic nanocomposite can be dried without being formed into a shape. The damp material or damp shapes may be stored for later use, or may be dried. The biomimetic nanocomposite, or shapes therefrom may be air-dried at ambient temperature, or may be dried using other means, such as a warm environment, or by using an enclosed space with a desiccant. The shaped or unshaped biomimetic nanocomposite, damp or dried, may be stored for later use, as the biomimetic nanocomposite is stable in normal atmosphere. Additionally, products may later be cut or shaped from the unformed and unshaped biomimetic nanocomposite.

Optionally, other components or additives, such as described earlier in this application, may be added to the biomimetic nanocomposite. The components may be added during the process, and at any stage, from the initial step to the last step. In addition, the other components may be added to the final biomimetic nanocomposite, whether damp or dry, and whether unformed or formed.

A polymerization matrix may be formed by using a gelatin as an embedding media for the mineralization of hydroxyapatite nanocrystals, adding a polymer, and adding a crosslinking agent. Preferably, these steps will be conducted sequentially, with mixing to obtain uniform dispersion at each step. In some cases, however, the polymer and crosslinking agent may be added simultaneously, or near-simultaneously. Whether step-wise or combined, the polymer and crosslinking agent may be added quickly, or over a period of time. The components may be added by hand, by equipment such as a buret, or by using a pump or other automatic device.

### EXAMPLES

### Example 1

### Component Preparation

The starting materials used were CaCO₃ (Alkaline analysis grade, Aldrich, USA), H₃PO₄(AP grade, Aldrich, USA) and Gelatin (Unflavored, Natural Foods Inc., Canada). Pure Ca(OH)₂ was obtained through the hydration of CaO. CaCO₃ was calcined at 1150° C. for 3 hours, driving off CO₂ from the material, leaving CaO. The CaO hydration was then carried out at 250° C. using 3 times the stoichiometric amount of ion-exchanged, double-distilled water. The final Ca(OH)₂ content and quality were determined by measuring the dry weight of the resulting material, after the material was stored at 120° C. for 3 h. 0.1994 mol (14.7741 grams) of dried Ca(OH)₂ was dissolved into DD water for use later in the process, making 2 liters of a 0.0997 M Ca(OH)₂ solution.

A reaction flask was prepared with a magnetic stirrer and temperature control. In order to increase uniformity, the gelatin powders were dissolved in an aqueous solution of H₃PO₄. Gelatin and phosphoric acid were used in amounts sufficient to reach 0.03 mmol gelatin and 59.76 mmol H₃PO₄ in an aqueous solution. The gelatin was added to the phosphoric acid solution and mixed for about 6 hours.

### Example 2

### Co-Precipitation

The HAp-GEL composite slurry was prepared by the simultaneous titration method using peristaltic pumps (Masterflex, Cole-Parmer, USA), into a reactor set up with temperature control via a water bath (Boekel, USA) and a pH controller (Bukert 8280H, Germany). A teflon-coated stainless steel reactor was used, and the teflon coating prevented leaching contamination by the acid from the container. The temperature of the water bath was set to 38° C., and was digitally controlled to within 0.1° C. The pH target was set to 8.0, and controlled to within 0.1 pH through addition of the component streams. The two component streams were as described above in Example 1. The amounts of the components streams to be used were calculated to make 10 grams of HAp-GEL composite. The two component streams were gradually added to the reaction vessel through peristaltic pumps. After the co-precipitation reaction, the total volume was adjusted as 4 liters.

As the component streams were added, the co-precipitation began to occur. The hydroxyapatite formed on the GEL matrix by the co-precipitation. The HAp nanocrystals appeared as needle shaped crystals. The dynamic energy supplied by high speed stirring during the co-precipitation process assisted in obtaining uniformly developed needle-shaped particles of HAp in the GEL matrix. The temperature was maintained as described above, with a target set point of 38° C. After the components were added with agitation, the stirring was stopped. The mixture was allowed to rest, static. After 24 hours of aging, the slurry was collected by passing through a glass filter using vacuum filtration, and washed 5 times with double distilled water. This slurry is needed for the further reaction between GEL and HAp.

### Example 3

### Composition

A teflon-lined stainless steel reactor was setup with a magnetic stirrer and temperature control using a water bath. The temperature target was set to 37° C.

All of the HAp/GEL composite slurry (from Example 2) was added to the reactor. Then, an aqueous mixture of 2.0 grams polyacrylic acid was added into the reactor using a peristaltic pump under conditions of vigorous stirring. This began forming the composition matrix.

After 30 minutes of vigorous stirring, an aqueous mixture of 1.0 gram of glutaraldehyde (GA) was added as a crosslinking agent. Upon addition, a crosslinking reaction began, forming a matrix structure including the HAp/GEL composite, the polymer, and the crosslinking agent.

After about 15 minutes of mixing, agitation and temperature control was stopped. The resulting material was collected using vacuum filtration. The collected composite was then dried at ambient temperature for about 2 days. This formed a dry solid that was stable in atmosphere.

### Example 4

### Composition

A composition was prepared following the steps as described above in Example 1 and Example 2. Then, the steps of Example 3 were followed, with the exceptions that 2.0 grams of polyvinyl acetate was used instead of 2.0 grams of polyacrylic acid, and 1.0 grams of EDGE was used instead of 1.0 grams of GA. The reaction conditions, timing, and steps followed were otherwise the same as in Example 3.

### Example 5

### Water Immersion Testing

A water immersion test was conducted on the composition of Example 3, in a manner similar to water immersion testing for skin or other biotexture. A sample of the biomimetic nanocomposite was immersed in an enclosed bottle of double distilled water for about 72 hours. An aquilot of water was then tested for PAA using a suitable test method. The presence of PAA in water may be confirmed using FT-IR (liquid sample) testing or by gas chromatography (GC). Using FT-IR, a negligible amount of the PAA component was detected. Thus, it is believed that most of PAA was chemically bonded to the HAp/GEL composite through the cross-linkage reaction.

### Example 6

### Gelatin Variation

The steps described in Example 1 were followed, with the exception that the amount of gelatin dissolved in solution in Example 1 was modified each time. For example, the composition formed using a 0.03 mmol gelatin solution was called HAp-GEL3. The first column of TABLE 1 summarizes various gelatin concentrations used to form compositions.

The steps described in Example 2 were followed, with the exception that the gelatin component was formed using various concentrations of gelatin. The names of the resulting HAp-GEL composites are shown in column 2 of TABLE 1.

TABLE 1
Gelatin Concentrations - Hydroxyapatite
Compositions & Biomimetic Nanocomposites

| Gelatin Solution Concentration | HAp-GEL Composition |
|---|---|
| 0.03 | HAP-GEL3 |
| 0.05 | HAP-GEL5 |
| 0.10 | HAP-GEL10 |
| 0.15 | HAP-GEL15 |
| 0.20 | HAP-GEL20 |
| 0.30 | HAP-GEL30 |
| 0.40 | HAP-GEL40 |

These are alternative HAp-GEL composites that may be used as the HAp-GEL composite slurry at the start of Example 3, as discussed above.

### Example 7

### Co-Precipitation Temperatures

A series of co-precipitation experiments controlled at different temperatures led to the conclusion that the optimal temperature for the co-precipitation was greater than about 37° C. and less than about 48° C. Samples were synthesized using co-precipitation temperatures of 38° C., 47° C., 65° C., and 80° C. Thus, the samples were taken for analysis after the steps described in Examples 1 and 2 were completed, with the variation being the temperature of the co-precipitation reaction.

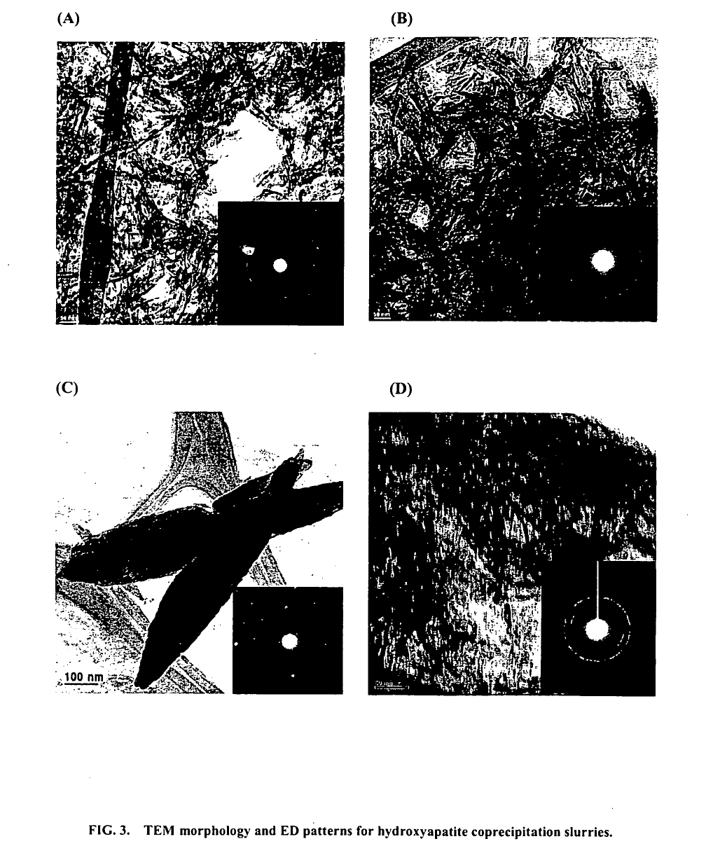

FIG. 3 shows TEM morphology and ED patterns for prepared samples, obtained using a JEOL 1210 transmission electron microscope. The samples were made using temperatures of 47° C. (A), 65° C. (B), 80° C. (C), and 38° C. (D). The scale bars in (A), (B), (C), and (D) indicate 50 nm, 50 nm, 100 nm, and 50 nm respectively. Samples A, B, and C were taken from HAp-GEL composition HAP-GEL3. Sample (D) was prepared from HAp-GEL composition HAP-GEL5.

Sample (D), the sample prepared at 38° C. demonstrated the preferred orientation of HAp crystals along the c-axis of the GEL molecule. When the temperature was increased above 48° C., the residual content of the GEL in the formed composite greatly decreased, but the needle-shaped crystal of HAp greatly increased in size with the increase of temperature. Additionally, alignment of the crystals become less aligned and more random. In the temperature range between 37° C. and 48° C., a good composite of HAp/GEL was obtainable, along with good orientation. When the temperature was decreased below 37° C., the residual content of the GEL in the formed composite greatly increased, but the needle shaped crystal of HAp were both slow and incomplete in forming and developing.

### Example 8

### Composition Discs

The steps described in Examples 1-3 were followed, with the amounts of materials used as described in Examples 1-3, except as follows: 5 grams gelatin (forming a 0.05 mmol concentration of gelatin), 0.5 g polyacrylic acid, and 0.8 g glutaraldehyde. The resulting composition from the steps in Example 3 (up to the drying stage) was formed into a cylinder having a 5 mm diameter. After drying for two days at ambient temperature, the dried composition was sliced to make 1 mm thick discs using a slow speed diamond saw.

### Example 9

### Chinese Hamster Ovary (CHO) Cells

Several discs from example 8 were obtained and placed in a 35 mm Petri dish.

CHO-K1 cells expressing Enhanced Green Fluorescent Protein (EGFP) were used for seeding on each composition disc. The cell line was obtained by transfecting CHO with plasmid pEGFP (Clontech) and selected on neomycine. The cells were seeded onto the composition discs at 4×10⁵ cells/cm² and cultured in Dulbecco's Modified Eagle Medium (DMEM), and 10% FB at 37° C. and 5% CO₂. Every 3 days, the samples (discs with seeded cells) were transferred to a new dish, and fresh media was added.

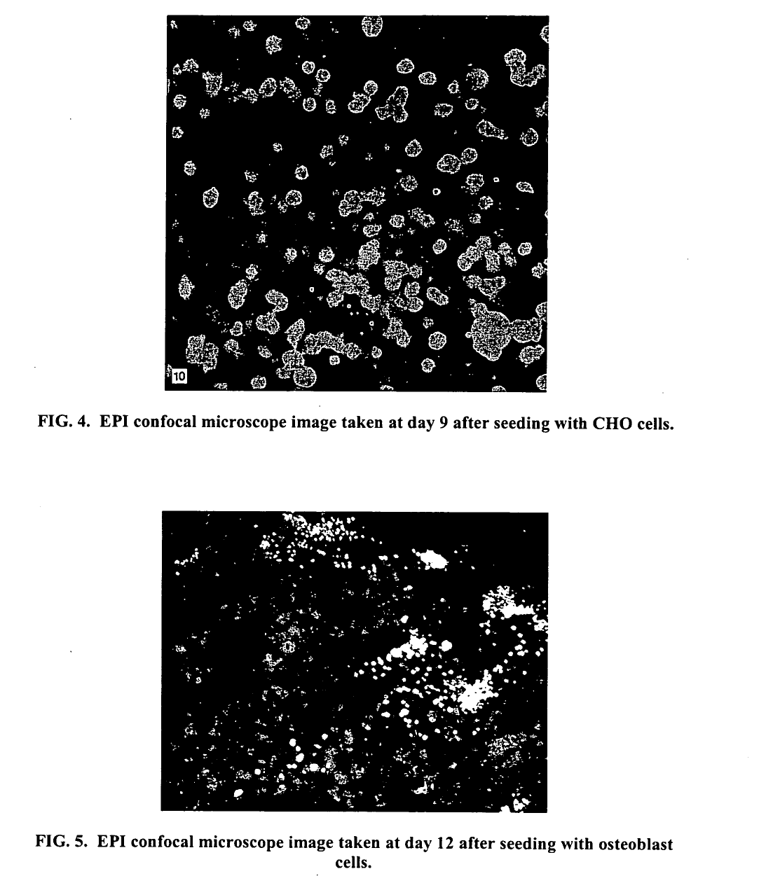

An EPI confocal microscope (Nikon-Diaphot, Nikon, Tokyo, Japan) was used to observe cells attached to the material. Excitation wavelength and emission wavelengths were 488 nm and 530 nm, respectively. The sample was observed and photographed after 9 days, as shown in FIG. 4. As shown in the photo, the cells attached to the biomimetic nanocomposite material and continued growing.

### Example 10

### Osteoblasts

Several discs from example 8 was obtained and placed in a 35 mm Petri dish.

Each disc was seeded with 10,000 cells using human fetal osteoblasts which had been cultured in alpha-Minimal Essential Medium (αMEM), supplemented with 5% fetal bovine serum (FBS), and incubated at 34° C. in 5% CO₂ environment.

After 12 days, the sample was stained using an ELF 97 Endogenous Phosphatase Detection Kit (E6601, Molecular Probes), and the sample observed and photographed using an EPI confocal microscope (Nikon-Diaphot, Nikon, Tokyo, Japan). The excitation wavelength was 345 nm and the emitted fluorescence was visualized through a typical DAPI filter. The resulting sample is shown in FIG. 5. This example demonstrated that osteoblasts can get differentiated on the material.

### Example 11

### Bone Marrow Stem Cells

Several discs from example 8 were obtained and placed in a 35 mm Petri dish.

Cells at a density of 50,000 cells/cm² (equivalent to about 50% confluence) were seeded onto each disc in the 35 mm dish and incubated in an atmosphere containing 5% CO₂ at 37° C. At day 3, FDA-EB was added into the dish and incubated at 37° C. for 5 minutes.

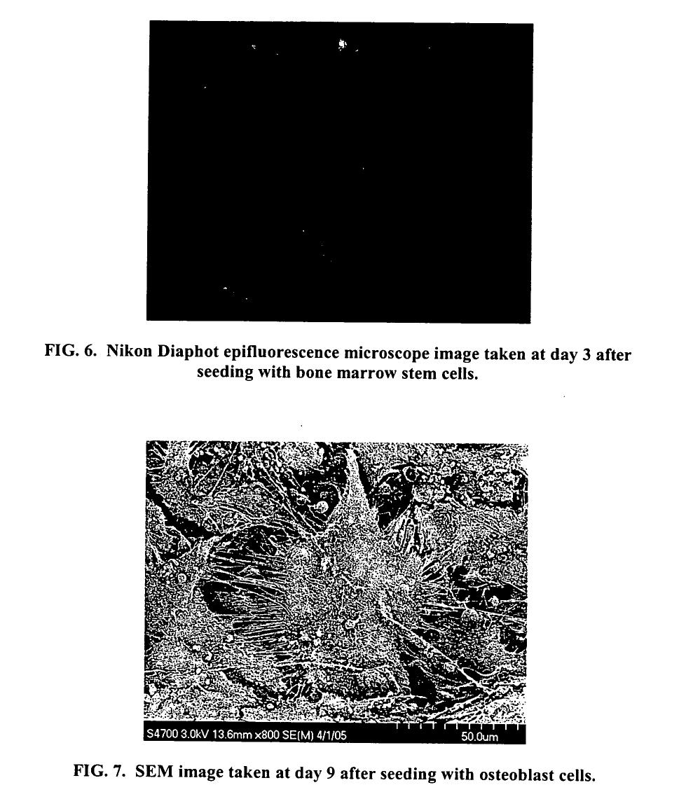

For FIG. 6, a fluorescent staining procedure with fluorescein diacetate (FDA) (Takasugi 1971) was used to assess the cell viability in situ. As a non-polar molecule, FDA passed through the cell membrane. The intracellular esterase hydrolyzed FDA to the polar green fluorescent fluorescein, which accumulated in the cytoplasm of the intact viable cells. Cells were visualized with a Nikon Diaphot epifluorescence microscope (Nikon, Melville, N.Y.) connected to a confocal laser scanning system (Multiprobe 2001, Molecular Dynamics, Sunnyvale, Calif.). This demonstrated that bone marrow cells grew on the material.

### Example 12

### Osteoblasts

Several discs from example 8 were obtained and placed in a 35 mm Petri dish.

Each disc was seeded with 10,000 cells using human fetal osteoblasts which had been cultured in alpha-Minimal Essential Medium (αMEM), supplemented with 5% fetal bovine serum (FBS), and incubated at 34° C. in 5% CO₂ environment.

After 9 days, the cells were fixed using 2.5% glutaraldehyde and dehydrated for scanning electron microscope (SEM) observation. The SEM scanning of a sample is shown in FIG. 7, at the scale as shown at the bottom of the figure. This SEM image showed cell morphology and interaction between the osteoblast and the biomimetic nanocomposite material. There were linkages extended out of cells and attached to the materials, indicating a good compatibility of the material.

A number of embodiments of the invention have been described. Nevertheless, it will be understood that various modifications may be made without departing from the spirit and scope of the invention. Accordingly, other embodiments are within the scope of the following claims.

What is claimed is:

1. A biomimetic nanocomposite, comprising:
hydroxyapatite nanocrystals;
gelatin; and
polymer,
wherein the biomimetic nanocomposite is crosslinked.

2. The nanocomposite of claim 1, wherein the biomimetic nanocomposite is crosslinked via a crosslinking agent.

3. The nanocomposite of claim 1, wherein the components are nearly uniformly dispersed.

4. The nanocomposite of claim 1, wherein the components form a crosslinked, complex matrix structure.

5. The nanocomposite of claim 1, wherein the gelatin comprises phosphorylated gelatin.

6. The nanocomposite of claim 1, further comprising a growth factor.

7. The nanocomposite of claim 6, wherein the growth factor is BMP, TGF-β, VEGF, MGP, BSP, OPN, OCN, IGF-I, or Pro COL-α1.

8. The nanocomposite of claim 1, further comprising cells.

9. The nanocomposite of claim 8, wherein the cells are osteoblasts, osteoclasts, or osteocytes.

10. The nanocomposite of claim 8, wherein the cells are stem cells.

11. The nanocomposite of claim 1, wherein the nanocomposite is formed into a porous scaffold.

12. An article for use in tissue engineering, wherein the article comprises the nanocomposite of claim 1.

13. An article for use as bone replacement, wherein the article comprises the nanocomposite of claim 1.

14. A method for producing a biomimetic nanocomposite, comprising:
mixing calcium hydroxide, phosphoric acid, and gelatin under aqueous conditions;
co-precipitating the mixture;
adding a polymer; and
adding a cross-linking agent.

15. The method of claim 14, wherein the mixing occurs at a temperature from about 37° C. to about 48° C.

16. The method of claim 14, wherein the pH of the mixture is maintained at about 7.5 to about 8.5.

17. The method of claim 14, wherein the number of moles of calcium hydroxide present in the mixture is about 1.5 to about 2.0 times the number of moles of phosphoric acid present in the mixture.

18. A method of using a biomimetic nanocomposite, comprising:
implanting an article comprising a biomimetic nanocomposite, wherein the biomimetic nanocomposite comprises: hydroxyapatite nanocrystals; gelatin; and
polymer, and wherein the biomimetic nanocomposite is crosslinked.

19. A method of bone regeneration, comprising using the nanocomposite of claim 1.

20. A method of cartilage regeneration, comprising using the nanocomposite of claim 1.
